(19) United States
(12) Patent Application Publication
(10) Pub. No.: US 2011/0160547 A1
(43) Pub. Date: Jun. 30, 2011

(54) WIRELESS TRANSMISSION DEVICE FOR PHYSIOLOGICAL INFORMATION

(76) Inventor: Chien-Yuan Yang, Yonghe City (TW)

(21) Appl. No.: 12/649,043

(22) Filed: Dec. 29, 2009

(51) Int. Cl. A61B 5/00; H04B 7/00

(52) U.S. Cl. 600/301; 455/41.2

(57) ABSTRACT

A wireless transmission device for physiological information is carried personally by a user and is attached on skin to measure the user's physiological condition by touching the skin. Related physiological signals are continuously and automatically transmitted to other monitoring device through an automatic wireless transmission unit in the wireless transmission device. Therefore, the user's physical condition can be monitored at any time and the related physiological information can be provided to a medical facility or other monitoring facility for understanding and tracking.

### BACKGROUND OF THE INVENTION

a) Field of the Invention

The present invention relates to a physiological information measuring apparatus and more particularly to a wireless transmission device for physiological information, which can monitor a user's physiological information at any time and then automatically transmit the physiological information to a server, such that the user's physiological condition can be kept track of at any time.

b) Description of the Prior Art

As a fast change of societies, a quality of life and material enjoyment of a modern person are quite enriched. Therefore, a dieting habit and a living style are relatively luxury. Due to the change of social patterns, elders cannot be taken care of comprehensively at all time by family members or personal nurses, and the elders cannot be even looked after if they go out for shopping or take a walk. On the other hand, when an accident happens, such as falling down, having a stroke, getting a fever or having a shock, if it is not found in time, then it is possible to cause a danger to life safety.

Accordingly, how to solve the aforementioned issues and shortcomings of the prior art is a direction of research and development for improvement by the present inventor and related vendors.

### SUMMARY OF THE INVENTION

The present invention is to provide a wireless transmission device for physiological information, which can be carried personally by a user and uses a measuring unit in the wireless transmission device to measure a physiological condition of the user by touching skin, wherein an interior of the wireless transmission device is provided with an automatic wireless transmission unit which continuously and automatically transmit related physiological signals that have been measured, the automatic wireless transmission unit will transmit these physiological signals to a wireless transmitter for analysis, and then the wireless transmitter will transmit wirelessly the analyzed physiological signals to at least one monitoring device. As a result, the user's physical condition can be monitored at any time and the related physiological information can be provided to a medical facility or other monitoring facility for understanding and tracking.

Another object of the present invention is to provide a wireless transmission device for physiological information, which is provided with a simple overall structure to facilitate carrying by the user.

Still another object of the present invention is to provide a wireless transmission device for physiological information, which is provided with an easy operation method and can track the user's physiological condition at any time.

To enable a further understanding of the said objectives and the technological methods of the invention herein, the brief description of the drawings below is followed by the detailed description of the preferred embodiments.

### BRIEF DESCRIPTION OF THE DRAWINGS

FIG. 1 shows a block diagram of a preferred embodiment of the present invention.

FIG. 2 shows a schematic view of a usage state of the preferred embodiment of the present invention.

### DETAILED DESCRIPTION OF THE PREFERRED EMBODIMENTS

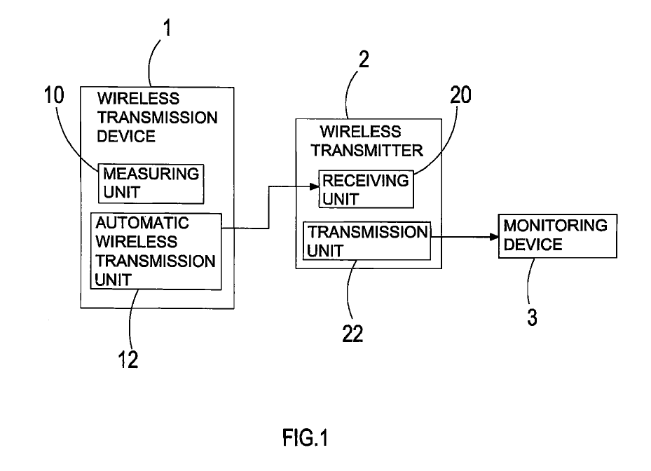

Referring to FIG. 1, it shows a block diagram of a preferred embodiment of the present invention. As shown in the drawing, the present invention is a wireless transmission device for physiological information. The wireless transmission device 1 of the present invention can be carried personally by a user and uses a measuring unit 10 in the wireless transmission device 1 to measure his or her physiological condition by touching skin and measuring a micro-current produced from the skin, wherein an interior of the wireless transmission device 1 is provided with an automatic wireless transmission unit 12 which continuously and automatically transmit related physiological signals that have been measured, and the automatic wireless transmission unit 12 will transmit these physiological signals to a wireless transmitter 2. As an interior of the wireless transmitter 2 (e.g., a cell phone) is provided with a receiving unit 20 (e.g., a Bluetooth or a signal connection line) and a transmission unit 22, the receiving unit 20 in the wireless transmitter 2 can analyze the received physiological signals through a data processing chip (not shown in the drawing) in the wireless transmitter 2, and then the analyzed physiological signals are transmitted wirelessly to at least one monitoring device 3 (e.g., a cell phone or a server) through the transmission unit 22. On the other hand, the physiological signals can be also computed first through a data processing chip (not shown in the drawing) in the wireless transmission device 1 and then transmitted to the wireless transmitter 2.

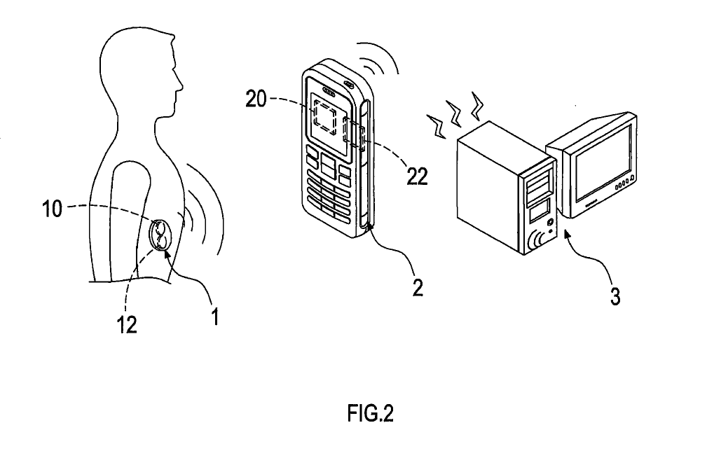

Referring to FIG. 2, it shows a schematic view of a usage state of the preferred embodiment of the present invention. As shown in the drawing, the user first installs the wireless transmission device 1 on his or her body and attaches to the skin. At this time, the measuring unit 10 will detect continuously the micro-current produced by the body, and the physiological signals detected from the micro-current will be continuously and wirelessly transmitted to the wireless transmitter 2 through the automatic wireless transmission unit 12. On the other hand, this wireless transmitter 2 can be put on a piece of clothes, a back bag or a location of hanging, depending upon the user's preference.

When the receiving unit 20 in the wireless transmitter 2 (e.g., a cell phone) receives the physiological signals transmitted by the automatic wireless transmission unit 12, the physiological signals will be further transmitted to at least one monitoring device 3 (e.g., another cell phone or a server) wirelessly using the transmission unit 22. Accordingly, by the aforementioned devices and means of use, the user's physiological information can be kept track of at any time.

It is of course to be understood that the embodiments described herein is merely illustrative of the principles of the invention and that a wide variety of modifications thereto may be effected by persons skilled in the art without departing from the spirit and scope of the invention as set forth in the following claims.

What is claimed is:

1. A wireless transmission device for physiological information being directly worn on a user and touching skin, comprising a measuring unit to detect a physiological condition of the user; an interior of the wireless transmission device being provided with an automatic wireless transmission unit which continuously and automatically transmit related physiological signals that have been measured, the automatic wireless transmission unit transmitting these physiological signals to a wireless transmitter which is provided with a receiving unit and a transmission unit, the receiving unit analyzing the received physiological signals, whereas the analyzed physiological signals being transmitted wirelessly to at least one monitoring device through the transmission unit.

2. The wireless transmission device for physiological information according to claim 1, wherein the receiving unit is a Bluetooth or a signal connection line.

3. The wireless transmission device for physiological information according to claim 1, wherein the monitoring device is a cell phone or a server.
